(19) United States
(12) Patent Application Publication
(10) Pub. No.: US 2008/0220069 A1
(43) Pub. Date: Sep. 11, 2008

(54) Long Acting Injectable Crystal Formulations of Estradiol Metabolites and Methods of Using Same

(75) Inventor: S. Dean Allison, Fort Collins, CO (US)

Correspondence Address: THE MCCALLUM LAW FIRM, P. C., 685 BRIGGS STREET, PO BOX 929, ERIE, CO, 80516, US

(73) Assignee: PR PHARMACEUTICALS, INC., Fort Collins, CO (US)

(21) Appl. No.: 11/663,003

(22) PCT Filed: Sep. 13, 2005
(86) PCT No.: PCT/US05/33158

§ 371 Date: Mar. 13, 2007

Related U.S. Application Data
(60) Provisional application No. 60/609,632, filed on Sep. 13, 2004.

(51) Int. Cl. A61K 31/56; C07J 1/00; A61K 9/14

(52) U.S. Cl. 424/489; 552/625; 552/618; 514/182

(57) ABSTRACT

The present invention provides sustained release formulations of estradiol metabolites whereby the in vivo pharmacokinetics are manipulated by a method selected from the group consisting of chemical modification, crystal packing formation, particle size or a combination thereof. Such compositions are useful in the long-term treatment of a wide variety of diseases.

### FIELD OF THE INVENTION

This invention relates generally to sustained release formulations of estradiol metabolites in crystal form, whereby the pharmacokinetics are controlled by crystal structure and particle size.

### BACKGROUND OF THE INVENTION

Estradiol is converted into different derivatives through metabolic processes in vivo. Two particular types of metabolites are the catecholestrogens and the methoxyestradiols. The catecholestrogens, 2-hydroxyestradiol and 4-hydroxyestradiol, are created by hydroxylation of estrogen via cytochrome P450 enzymes. The catecholestrogens can be methylated by catechol-O-methyl transferase to create the methoxyestradiols, 2-methoxyestradiol and 4-methoxyestradiol.

Estradiol metabolites have been reported to have an effect on a number of cellular processes. Estradiol metabolites apparently inhibit angiogenesis and the polymerization and organization of tubulin in actively growing cells (Brueggemeier, R. W., et al., 2001: Pribluda, V. S. et al., 2000; U.S. Pat. No. 5,504,074; International patent publication No. WO0/35865) and induce apoptosis in some cells. (Schumacher, G., et al., 2001; U.S. Pat. No. 5,958,892; Wang, S. H., et al., 2000). In addition, 2-hydroxyestradiol and 2-methoxyestradiol appear to affect cholesterol levels in ovarectomized rats and to inhibit adipose cell proliferation in culture (Liu, D., et al., 1998; Pico, C., et al., 1998), while 2-hydroxyestradiol apparently decreases the effects of obesity, metabolic syndrome, and vascular and renal dysfunction in obese rats (Dubey, R. K., et al., 2001). Estradiol metabolites are also reported to be beneficial in the treatment of end-stage renal disease and asthma (Dubey, R. K. et al., 2001; U.S. Pat. No. 6,200,966). Additionally, estradiol metabolites appear to be effective antifungal agents (U.S. Patent App. No. 2001/44432).

Beneficial effects of estradiol metabolites have been reported for cancer treatment (International patent application No. WO01/70093). 2-methoxyestradiol appears to decrease the growth of lung cancer cells in culture when administered with wild-type p53 (Huober, J. B., et al., 2000; Kataoka, M., et al., 1998), to inhibit the growth of human pancreatic (Schumacher, G., et al., 1999) and prostate cancer cells (Kumar, A. P., et al., 2001; Qadan, L. R., et al., 2001) and to be toxic to osteosarcoma cells (Maran, A., et al., 2002). 2-methoxyestradiol was also reported to decrease the growth rate of neuroblastoma cells and tumors of the pituitary gland (Banerjeei, S. K., et al., 2000; Wassberg, E., 1999). Estradiol metabolites apparently increase the intracellular accumulation of superoxide anions in rapidly dividing cells (International patent application No. WO02/03979) and enhance the effects of existing cancer treatments, such as radioimmunotherapy (Amorino, G. P., et al., 2001; Kinuya, S., et al., 2001).

Two main strategies exist to prolong the duration of exposure to rapidly metabolized drugs, particularly steroids. The first is to increase plasma circulation time by chemically modifying the steroid with organic acids to form steroid ester prodrugs. After delivery, the steroid ester bond is cleaved to form the parent compound by endogenous enzymes. Physical and chemical properties imparted to the steroid by the organic acid, or other modifying compound, govern the rate at which the parent compound is released from its prodrug form. In this way, the plasma circulation time can be increased in a controlled manner. Steroid esters are routinely dissolved and injected in an oil vehicle for sustained delivery. However, the duration of oil based depot injections is limited to a few days, in most cases. The second strategy to achieve sustained exposure to estradiol metabolite therapeutics is to incorporate the estradiol metabolite, or estradiol metabolite prodrug into a sustained release delivery device. Such devices may include injectable or implantable osmotic pumps, liposomes, solid lipid particles, or diffusion-mediated inert or biodegradable matrices or depots.

There are several disadvantages surrounding the manufacture and administration of drugs formulated in delivery devices. First, administration of the device may require a surgical procedure. This may be particularly disadvantageous in cases where long-term treatment is required. Second, the added bulk of the device to the formulation may limit the amount of the active pharmaceutical ingredient that may be administered without undue patient discomfort. Third, the physical form of the estradiol metabolite encapsulated within the device may be unstable, and susceptible to chemical degradation, limiting the types of devices capable of being used to formulate the drug. The drawbacks are particularly true in the case of estradiol metabolites, which have generally low solubility and have applications for the treatment of chronic diseases.

Leonard, in U.S. Pat. No. 5,688,519 (1997) describes compositions and methods resulting in the production of thermally fused cylindrical steroid pellets approximately 6-7 millimeters in length and 2.4 millimeters in diameter. Pellets consisting of fused norethindrone released drug into water over a period of months. While sustained release of steroid compounds can be achieved with this type of formulation, subcutaneous implantation of the pellets requires more skill than a typical liquid injection and the procedure involves elevated patient discomfort.

A second class of micronized crystal suspensions for injection are formulated to release drug over a period of weeks. For example, according to package inserts, methylprednisolone acetate (Depo-Medrol®) is dosed at two week intervals for systemic action, and the contraceptive medroxyprednisolone acetate (Depo-Provera®) is injected every thirteen weeks. Particle size distribution for the two products is approximately the same, ranging from 0.4 to about 20 micrometers with the modes between 7 and 9 micrometers. The chemical modification to form acetate esters is the same for both products. These two water insoluble compounds have widely varying durations of action despite having the same chemical modification and comparable particle size distributions. In addition, U.S. Pat. No. 4,794,119 demonstrated measurable blood levels for an injectable crystal suspension of a progestational steroid ester for 4 weeks in primates. The particle size distribution of the steroid in the formulation ranged between 3 and 100 micrometers with the mode at approximately 24 micrometers. Thus, steroids classified as insoluble in water show varying durations in physiological environments after injection. Some degree of control over the in vivo pharmacokinetics may be achieved by altering particle size and/or chemical modification. However, necessary modifications to the chemical composition of the steroid and to the size distribution of the crystalline particles that are effective in achieving the desired in vivo pharmacokinetics are not obvious, and must be discovered especially for each compound.

Estradiol metabolites may be useful in the treatment or prevention of a variety of diseases. Unfortunately, naturally occurring estradiol metabolites have poor oral bioavailability and a short half-life, and the beneficial effects appear to be tied to a prolonged period of treatment. A need exists for pharmaceutical formulations of estradiol metabolites, which increase the duration of action of the metabolites without necessitating frequent administrations, which would be undesirable in both animal and human patients. A particular need exists for compositions and methods for sustained release injectable crystal suspensions of estradiol metabolites, including formulations with desired in vivo pharmacokinetics that are controlled by adjusting the chemical form and crystal structure, as well as the particle size distribution of the steroid molecules. The development of a sustained release system for estradiol metabolites would provide an improved therapeutic option for treatment of a wide variety of veterinary and human diseases.

### SUMMARY OF THE INVENTION

The present invention relates to sustained release formulations of estradiol metabolites and methods of making and using the same.

An object of the present invention is to manipulate the solubility and in vivo pharmacokinetic properties of crystalline estradiol metabolites, estradiol metabolite analogs, or estradiol metabolite prodrugs in order to prepare sustained release formulations that can be administered by a simple injection. Such formulations would provide the desired long-term release characteristics available in a polymer matrix without the limiting added bulk of the release-regulating device. In addition, formulations consisting of crystalline compounds are likely to be highly stable compared to the amorphous or solution states of the compound contained in controlled release devices. Furthermore, crystal suspension formulations of estradiol metabolites, estradiol metabolite analogs, and estradiol metabolite prodrugs are likely to be simpler and less expensive to manufacture than polymer matrix devices.

Ideally, injectable sustained release dosage forms would be highly stable, would be simple enough to permit patient self-administration, and would minimize the volume of material injected per dose, thereby minimizing patient discomfort. Accordingly, it is an object of the present invention to provide injectable sustained release formulations based on controlled in vivo pharmacokinetics of estradiol metabolites, estradiol metabolite analogs, or estradiol metabolite prodrugs from the solid crystalline state. Embodiments of the present invention provide sustained release formulations in which the in vivo pharmacokinetic properties of the drug are controlled by manipulating individually, or in combination, the chemical form of the drug, the crystal polymorph, the particle size, and the weight of the drug. Such alterations may affect many properties, such as the dissolution rate.

In certain embodiments, the present invention provides compositions of matter or methods in which estradiol metabolite compounds are chemically derivatized or altered in order to increase or decrease the solubility of the altered compound in aqueous media relative to that of the parent compound. The chemical alterations may be reversible upon dissolution in physiological environments, so as to regenerate the original estradiol metabolite. Alternatively, the chemical modification may be irreversible in physiological environments. In such cases, the basic nature of the pharmacological activity of the altered estradiol metabolite will remain qualitatively intact. Such prodrugs may be ester derivatives of estradiol metabolites. Derivatives include but are not limited to dicarboxylic acid compounds, diacids, polar compounds, and ionic compounds.

In one embodiment, estradiol metabolites are catecholestrogens or methoxyestradiols. Estradiol metabolites of use in the present invention may be selected from the group consisting of 2-methoxyestradiol, 2-hydroxyestradiol, 4-methoxyestradiol, and 4-hydroxyestradiol. In a certain embodiment, the ester derivative of an estradiol metabolite is selected from the group consisting of 3-benzoyl-2-methoxyestradiol; 17-benzoyl-2-methoxyestradiol; 17-acetyl-2-methoxyestradiol; 3-acetyl-2-methoxyestradiol; 3,17-dibenzoyl-2-methoxyestradiol; 3,17-diacetyl-2-methoxyestradiol; 3-benzoyl-4-methoxyestradiol; 17-benzoyl-4-methoxyestradiol; 17-acetyl-4-methoxyestradiol; 3,17-dibenzoyl-4-methoxyestradiol; 3,17-diacetyl-4-methoxyestradiol; 2-benzoyl-2-hydroxyestradiol; 17-benzoyl-2-hydroxyestradiol; 17-acetyl-2-hydroxyestradiol; 3-acetyl-2-hydroxyestradiol; 3,17-dibenzoyl-2-hydroxyestradiol; 3,17-diacetyl-2-hydroxyestradiol; 2,3-dibenzoyl-2-hydroxyestradiol; 2,17-dibenzoyl-2-hydroxyestradiol; 2,17-diacetyl-2-hydroxyestradiol; 2,3-diacetyl-2-hydroxyestradiol; 2,3,17-tribenzoyl-2-hydroxyestradiol; 2,3,17-triacetyl-2-hydroxyestradiol; 3-benzoyl-4-hydroxyestradiol; 17-benzoyl-4-hydroxyestradiol; 17-acetyl-4-hydroxyestradiol; 3-acetyl-4-hydroxyestradiol; 3,17-dibenzoyl-4-hydroxyestradiol; 3,17-diacetyl-4-hydroxyestradiol; 3,4-dibenzoyl-4-hydroxyestradiol; 4,17-dibenzoyl-4-hydroxyestradiol; 4,17-diacetyl-4-hydroxyestradiol; 3,4-diacetyl-4-hydroxyestradiol; 3,4,17-tribenzoyl-4-hydroxyestradiol; 3,4,17-triacetyl-4-hydroxyestradiol, and the like.

The estradiol metabolite, estradiol metabolite prodrug, or estradiol metabolite analog of the present invention may be manufactured by any method known in the art into a desired crystal structure, so as to dissolve in physiological media in a desired time frame, ranging from days to months. Exemplary methods used to achieve this purpose include, but are not limited to, thermal treatments, spray drying, or antisolvent precipitation.

In other embodiments of the present invention, estradiol metabolite, estradiol metabolite analogs, or estradiol metabolite prodrugs with a desired crystal packing structure may be treated by annealing, grinding, milling, or other such method to achieve particles with a defined size distribution, in order to control in vivo pharmacokinetics, such as dissolution rate of the composition. In further embodiments, particle preparations with different size distributions may be blended together to achieve the desired in vivo pharmacokinetics.

Embodiments of the present invention include chemical modification, crystal structure, particle size that may further control the in vivo pharmacokinetic properties of the estradiol metabolite, estradiol metabolite analog, or estradiol metabolite prodrug.

The present invention provides aqueous diluents used to suspend drug crystals to permit intramuscular, intraperitoneal, intraocular, subgingival, or subcutaneous injection of the drug. The diluent may contain any salt, buffer, osmolyte, or other compound known and practiced in the art to achieve isotonicity. In further embodiments, the suspending agent contains a surfactant used to aid in the wetting and suspending of the crystals.

The final formulation may consist of estradiol metabolite or estradiol metabolite derivative crystals suspended in a diluent. Alternatively, the estradiol metabolite or estradiol metabolite derivative crystals may be supplied with diluent packaged, in the same or a separate container.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION OF THE INVENTION

The present invention may be understood more readily by reference to the following detailed description of particular embodiments of the invention and the Examples included therein.

Before the present compounds, compositions, and/or methods are disclosed and described, it is to be understood that this invention is not limited to specific synthetic methods, specific reagents or to laboratory or manufacturing techniques, as such may, of course, vary, unless it is otherwise indicated. It is also to be understood that the terminology used herein is for the purpose of describing particular embodiments only and is not intended to be limiting.

### DEFINITIONS

For the purposes of the present invention, the following terms shall have the following meanings:

The term “analog” and its cognates refer to any molecule that demonstrates estradiol metabolite activity. Such molecule may be a synthetic analog, fragment of estradiol metabolite or endogenous biological molecule other than an estradiol metabolite capable of estradiol metabolite-like activity. In sum, an estradiol metabolite analog refers to any molecule that demonstrates bioactivity similar or greater than an estradiol metabolite itself.

For the purposes of the present invention, the term “prodrug” refers to any modification of an estradiol metabolite, including a physical or chemical alteration that results in an increased plasma circulation time, or altered solubility in tissue fluids. The chemical modification or modifications to the drug may be reversible upon administration to an individual. The end product may include an estradiol metabolite, another estradiol prodrug or an estradiol analog.

For the purposes of the present invention, the term “estradiol metabolite” includes any molecule that originates from estradiol; and any molecule derived from an estradiol metabolite, an estradiol prodrug or an estradiol analog.

For the purposes of the present invention, the term “drug” may refer to any estradiol metabolite, any estradiol metabolite analog, or estradiol metabolite prodrug.

For the purposes of the present invention, the term “sustained release” may refer to release of estradiol metabolite over the course of a few days to a month or more.

Moreover, for the purposes of the present invention, the term “a” or “an” entity refers to one or more than one of that entity; for example, “a prodrug” or “an estradiol metabolite” refers to one or more of those compounds, or at least one compound. As such, the terms “a” or “an”, “one or more” and “at least one” can be used interchangeably herein. It is also to be noted that the terms “comprising,” “including,” and “having” can be used interchangeably.

Furthermore, a compound “selected from the group consisting of” refers to one or more of the compounds in the list that follows, including mixtures (i.e. combinations) of two or more of the compounds. According to the present invention, an isolated or biologically pure compound is a compound that has been removed from its natural milieu. As such, “isolated” and “biologically pure” do not necessarily reflect the extent to which the compound has been purified. An isolated compound of the present invention can be obtained from its natural source, can be produced using molecular biology techniques or can be produced by chemical synthesis.

Finally, for the purposes of the present invention, the term “individual” means an animal or human of either gender.

Reference will now be made in detail to particular embodiments of the invention.

Naturally occurring estradiol metabolites have a short plasma half-life. Oral bioavailability is low, in part due to rapid hepatic metabolism. In addition, some indications that can be treated using estradiol metabolites, estradiol metabolite analogs, or estradiol metabolite prodrugs, such as diabetic nephropathy, cancer, obesity, or pulmonary hypertension, require prolonged administration of the drug(s). Development of estradiol metabolites, estradiol metabolite analogs, or estradiol metabolite prodrug dosage forms for delivery over extended time periods is a novel way to administer these particular therapeutics in a useful fashion. International patent application number PCT/US03/12727 describes compositions of estradiol metabolites in combination with a material providing for sustained release. The present invention is distinguished from the above in that, rather than a second material being responsible for controlling release of the drug, the in vivo pharmacokinetic properties of estradiol metabolites from the crystalline state regulate the duration of release. Control of estradiol metabolite release may be exerted by altering the chemical and physical properties of the crystal.

From a crystal depot placed into a physiological environment, the systemic concentration of a drug at a given time depends on the overall solubility of the drug in the physiological milieu, the in vivo pharmacokinetics of the drug from the crystal, and the elimination rate of the drug. One strategy to achieve sustained release of estradiol metabolites, estradiol metabolite analogs, or estradiol metabolite prodrugs, is to manipulate the limited aqueous solubility and finite dissolution rates in tissue fluids of such compounds from the crystalline state. Steroids, including estradiol and estradiol metabolites, typically exhibit low solubility in aqueous media. The therapeutic utility of these compounds may be limited by the saturation concentration that is achievable in tissue fluids. However, these compounds may be chemically modified to control both the solubility and in vivo pharmacokinetics, so as to achieve the desired release profile.

### Chemical Modification of Estradiol Metabolites

In the present invention, a particular estradiol metabolite may be chosen to achieve the desired drug level and duration of action necessary to treat a particular condition. More than one estradiol metabolite may have antiproliferative activity against certain cell types. However, the physical and chemical properties of the estradiol metabolites, particularly the water solubility of these compounds can vary widely. For example, the water solubility of 2-hydroxyestradiol is approximately 100-fold higher than that of 2-methoxyestradiol. The solubility of estradiol metabolites in physiological environments may therefore affect both the rate of dissolution and the steady state concentration of the drug after injection. After injection of a crystal suspension, 2-hydroxyestradiol may be expected to have a much higher steady state concentration than an equal dose of 2-methoxyestradiol. Thus, the choice of a particular estradiol metabolite may be made as a first means of control to optimize the desired biological activity, dose level, and duration of action. Subsequent optimization of the dosage form may be implemented by chemically modifying the parent estradiol metabolite compound, and/or preparing crystals in the desired structure, and/or preparing crystals with the desired particle size distribution.

In certain embodiments, esters of estradiol metabolites are utilized to create prodrugs. The ester linkage stays intact during preparation and storage of the drug, only becoming vulnerable to hydrolysis after administration to a patient. Therefore, esters are optimal prodrugs because tissue fluids contain abundant endogenous esterases to catalyze hydrolysis of the linkage. Once hydrolysis occurs, only the active estradiol metabolite and a non-toxic biological compound remains, such as acetic acid or propionic acid, for example.

Esters may also be used to control solubility of the estradiol metabolites. Increased water solubility may be conferred by esterifying with succinic acid, for example. Other esters may improve solubility in a variety of other solvents, and may also allow for alterations in crystal packing geometry, which would control the release of the ester prodrug from the crystalline matrix and into the surrounding tissue fluids.

Esters of 2-methoxyestradiol of the present invention include, but are not limited to 3-benzoyl-2-methoxyestradiol, 17-benzoyl-2-methoxyestradiol, 17-acetyl-2-methoxyestradiol, 3-acetyl-2-methoxyestradiol, 3,17-benzoyl-2-methoxyestradiol and 3,17-diacetyl-2-methoxyestradiol.

Esters of 4-methoxyestradiol of the present invention include, but are not limited to 3-benzoyl-4-methoxyestradiol, 17-benzoyl-4-methoxyestradiol, 17-acetyl-4-methoxyestradiol, 3-acetyl-4-methoxyestradiol, 3,17-dibenzoyl-4-methoxyestradiol and 3,17-diacetyl-4-methoxyestradiol.

Esters of 2-hydroxyestradiol include, but are not limited to, 3-benzoyl-2-hydroxyestradiol, 17-benzoyl-2-hydroxyestradiol, 17-acetyl-2-hydroxyestradiol, 3-acetyl-2-hydroxyestradiol, 3,17-dibenzoyl-2-hydroxyestradiol, 3,17-diacetyl-2-hydroxyestradiol, 2,3-dibenzoyl-2-hydroxyestradiol, 2,17-dibenzoyl-2-hydroxyestradiol, 2,17-diacetyl-2-hydroxyestradiol, 2,3-diacetyl-2-hydroxyestradiol, 2,3,17-tribenzoyl-2-hydroxyestradiol and 2,3,17-triacetyl-2-hydroxyestradiol.

Esters of 4-hydroxyestradiol include, but are not limited to, 3-benzoyl-4-hydroxyestradiol, 17-benzoyl-4-hydroxyestradiol, 17-acetyl-4-hydroxyestradiol, 3-acetyl-4-hydroxyestradiol, 3,17-dibenzoyl-4-hydroxyestradiol, 3,17-diacetyl-4-hydroxyestradiol, 3,4-dibenzoyl-4-hydroxyestradiol, 4,17-dibenzoyl-4-hydroxyestradiol, 4,17-diacetyl-4-hydroxyestradiol, 3,4-diacetyl-4-hydroxyestradiol, 3,4,17-tribenzoyl-4-hydroxyestradiol and 3,4,17-triacetyl-4-hydroxyestradiol.

In certain embodiments, esters of all four estradiol metabolites may be organic acid derivatives of the original estradiol metabolite. In a particular embodiment, they include but are not limited to, esters of propionic acid, butyric acid, valeric acid, hexanoic acid, benzoic acid, acetic acid, propionic acid, butyric acid, stearic acid and other fatty acids.

Estradiol metabolites of use in the present invention may be selected from the group consisting of 2-methoxyestradiol, 2-hydroxyestradiol, 4-methoxyestradiol and 4-hydroxyestradiol. In another preferred embodiment, the ester derivative of an estradiol metabolite is selected from the group consisting of 3-benzoyl-2-methoxyestradiol; 17-benzoyl-2-methoxyestradiol; 17-acetyl-2-methoxyestradiol; 3-acetyl-2-methoxyestradiol; 3,17-dibenzoyl-2-methoxyestradiol; 3,17-diacetyl-2-methoxyestradiol; 3-benzoyl-4-methoxyestradiol; 17-benzoyl-4-methoxyestradiol; 17-acetyl-4-methoxyestradiol; 3-acetyl-4-methoxyestradiol; 3,17-dibenzoyl-4-methoxyestradiol; 3,17-diacetyl-4-methoxyestradiol; 3-benzoyl-2-hydroxyestradiol; 17-benzoyl-2-hydroxyestradiol; 17-acetyl-2-hydroxyestradiol; 3-acetyl-2-hydroxyestradiol; 3,17-dibenzoyl-2-hydroxyestradiol; 3,17-diacetyl-2-hydroxyestradiol; 2,3-dibenzoyl-2-hydroxyestradiol; 2,17-dibenzoyl-2-hydroxyestradiol; 2,17-diacetyl-2-hydroxyestradiol; 2,3-diacetyl-2-hydroxyestradiol; 2,3,17-tribenzoyl-2-hydroxyestradiol; 2,3,17-triacetyl-2-hydroxyestradiol; 3-benzoyl-4-hydroxyestradiol; 17-benzoyl-4-hydroxyestradiol; 17-acetyl-4-hydroxyestradiol; 3-acetyl-4-hydroxyestradiol; 3,17-dibenzoyl-4-hydroxyestradiol; 3,17-diacetyl-4-hydroxyestradiol; 3,4-dibenzoyl-4-hydroxyestradiol; 4,17-dibenzoyl-4-hydroxyestradiol; 4,17-diacetyl-4-hydroxyestradiol; 3,4-diacetyl-4-hydroxyestradiol; 3,4,17-tribenzoyl-4-hydroxyestradiol; 3,4,17-triacetyl-4-hydroxyestradiol.

Methods for synthesizing several esters of estradiol metabolites are known. (See, e.g., Japanese Patent No. 57,041,479 and 49,100,070).

The skilled artisan will realize that the compounds listed above are exemplary only and that many variations may be used, depending on the particular ester derivative created from a particular estradiol metabolite. Such variations are known in the art.

In further embodiments, compounds exhibiting estradiol metabolite activity but have little or no structural similarity to steroids, may be incorporated into injectable crystal depot formulations. Such estradiol analogs may themselves have the solubility and in vivo pharmacokinetic properties to permit sustained activity for a desired duration. In alternative embodiments, estradiol analogs may be chemically modified to form estradiol analog prodrugs. The function of the chemical modification to the estradiol analog parent compound is to achieve the purpose of modifying the solubility and in vivo pharmacokinetic properties of the estradiol analog so that sustained delivery of an appropriate concentration of the drug may be realized. The chemical modification may be reversible in physiological environments.

### Crystal Polymorphism of Estradiol Metabolites

Complex organic molecules, such as steroids, can form crystals in which the component molecules pack together in a finite variety of geometric orientations. Compounds capable of packing into a variety of geometries are said to be polymorphic. Packing geometry is commonly measured using x-ray crystallographic techniques. For a given compound, the number and strength of interactions between neighboring molecules will differ as the relative molecular orientation changes within a unit cell of the crystal. As a result, the energy required to release one molecule of the compound from the crystal lattice will increase or decrease depending on the packing geometry. This property, in part, controls the rate of dissolution of a compound into a solvent medium.

The packing geometry of a compound in the crystalline state can be controlled using a variety of processes well known in the art, and may include annealing of amorphous or crystalline material, spray drying, or antisolvent precipitation methods from solutions of the compound. The conditions under which the physical and chemical properties of an estradiol metabolite solution are changed can govern the packing orientation and particle size of newly formed crystals. For instance, recrystallization kinetics may be governed by the volume of a liquid non-solvent (e.g., water) for an estradiol metabolite, such as 2-methoxyestradiol, when an organic solution containing the metabolite is injected into the non-solvent reservoir. Other conditions, such as temperature, the solubility of the solvent in the non-solvent, and so on, can also influence crystal structure and particle size of recrystallized estradiol metabolites. Certain embodiments of the present invention provide compositions of estradiol metabolites, estradiol metabolite analogs, or estradiol metabolite prodrugs in which the packing geometry of the drug has been manipulated by any technique in order to aid in achieving the desired in vivo pharmacokinetics.

### Particle Size of Estradiol Metabolite Crystals

For any given packing geometry, in vivo pharmacokinetics of a compound from the crystalline state further depends on the specific surface area when the particles are in the micrometer size range (Martin, Physical Pharmacy 4^{th} ed. (1993) Williams and Wilkins, Baltimore, USA). Thus, for a given mass of crystalline particles with a smaller average size, more molecules will be present at the surface of the solid, increasing the likelihood of solvation over time. Particle size can be controlled using standard annealing, grinding, or milling methods known to practitioners of the art. The grinding, milling, or annealing process may be applied to a drug preparation with the intention of creating any desired particle size distribution. The particle size distribution may be manipulated to modify or adjust the overall rate of dissolution of the estradiol metabolite, estradiol metabolite analog, or estradiol metabolite prodrug. The milling process itself may be designed to create a particular distribution, or separate preparations of the drug with different particle size distributions may be processed by different milling procedures and mixed together to achieve the desired pharmacokinetic profile.

In certain embodiments, the grinding or milling process leads to a broad particle size distribution, where a certain proportion of the estradiol metabolite crystals are less than 5 μm in diameter. More rapid dissolution of the estradiol metabolite from the smaller particles results in an initial “burst” release of the drug that may be beneficial in the treatment of certain conditions.

### Suspending Liquid

The present invention provides aqueous diluents used to suspend drug crystals to permit intramuscular, intraperitoneal, intraocular, subgingival, or subcutaneous injection of the drug. The diluent may contain any salt, buffer, osmolyte, or other compound known and practiced in the art to achieve isotonicity. In further embodiments, the suspending agent contains a surfactant used to aid in the wetting and suspending of the crystals. The said surfactant may be selected from the group consisting of polyvinyl alcohol, polyvinylpyrrolidone tyloxapol, a poloxamer, a polyoxamine dextran lecithin a dialkylester of sodium sulfosuccinic acid, sodium lauryl sulfate, an alkyl aryl polyether sulfonate, a polyoxyethylene sorbitan fatty acid ester, polyethylene glycol, a mixture of sucrose stearate and sucrose distearate, C.sub. 18H.sub.37 CH.sub.2 (CON(CH.sub.3)CH.sub.2 (CHOH).sub.4 (CH.sub.20H).sub.2, carboxymethylcellulose calcium, carboxymethylcellulose sodium, methylcellulose, hydroxyethylcellulose, hydroxy propylcellulose, hydroxypropylmethylcellulose phthalate, noncrystalline cellulose, and isononylphenoxypoly(glycidol).

The following examples illustrate the preparation of 2ME crystals that vary in crystal shape and size distribution and an example of sustained plasma levels of 2ME after injection of crystalline 2ME.

### Treatment of Diseases

The compositions of the present invention may be used to treat any disease capable of responding to administration of estradiol metabolites.

One such disease capable of being treated with the compositions of the present invention includes metabolic syndrome. Treatment of metabolic diseases including obesity, diabetes mellitus, insulin resistance, hypertension and dyslipidemia alone or in combinations thereof frequently described as the metabolic syndrome or syndrome X Compositions of the present invention may also be utilized with any disease associated with endothelial dysfunction characterized by cellular proliferation, vascular remodeling, increased expression of vasoactive cytokines, increased deposition of extracellular matrix proteins, increased inflammation, and the like. Symptoms such as these are found in diseases such as hypertension, pulmonary hypertension, scleroderma and other collagen vascular diseases.

The compositions of the present invention may also be useful in the treatment of a disease selected from the group consisting of pulmonary hypertension, any disease associated with elevated endothelin-1 levels, diabetic nephropathy, nephrotoxicity and the like.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Preparation of Estradiol Metabolite Crystals

A 4% w/v solution of 2ME was prepared in a solvent consisting of 9.2% tetrahydrofuran, 61% methanol and 28% aqueous 6 molar hydrochloric acid. This solution was added dropwise to an equal volume of vigorously stirring water. The resulting solid was isolated by suction filtration, washed with water, and dried under vacuum. The resulting 2ME crystals were a mixture of large, hollow prisms greater than 500 μm in length by approximately 200 μm width, and cubic particles, ranging from approximately 50 μm down to approximately 500 nm square. The broad particle size range was intended to give a complex, biphasic pharmacokinetic profile upon injection.

### Example 2

### In Vivo Pharmacokinetics of an Estradiol Metabolite Crystal Preparation

The material generated in Example 1 was ground in a mortar and pestle, sieved through a 180 um screen, and the particle size distribution of the sieved material was measured on a Coulter LS13320 Particle Size analyzer. The volume averaged particle size was found to be 48.98±36.95 μm.

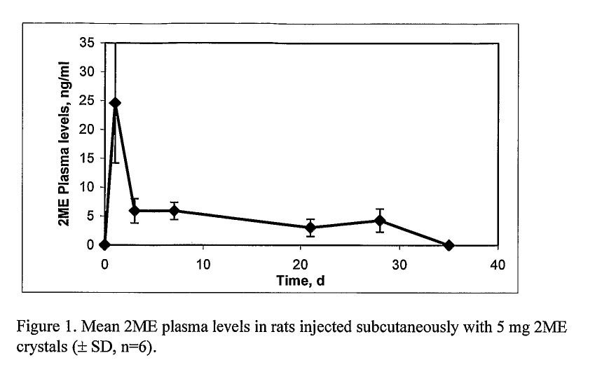

On day 0, animals in all treatment groups received 5 mg/rat of 2ME in 0.25 ml injection vehicle consisting of 2.0% w/v sodium carboxymethylcellulose containing 0.01% w/v Tween 80 and 0.1% w/v SDS by subcutaneous injection using a 1 cc syringe and a 1″, 20-gauge needle. On Day—3 blood was collected from all rats via the tail vein. On Days 1, 3, 7, 21, 28, and 35, all rats were bled via the lateral tail vein. Plasma samples were extracted, derivatized, and 2ME concentration was measured using a qualified gas chromatography-mass detection method. The pharmacokinetic profile is presented in FIG. 1. The plasma concentration profile of 2ME released from crystals with a broad size distribution is characterized by an initial burst followed by sustained blood levels of approximately 5 ng/ml between days 3 and 28. By day 35, 2ME blood levels had fallen to zero, indicating complete dissolution of the crystals.

### Example 3

### Esterification of Estradiol Metabolites to Change Water Solubility

2-methoxyestradiol was esterified to form 3-benzoyl-2-methoxyestradiol. The water solubility of 2ME is approximately 0.002 mg/ml at room temperature. The water solubility of the esterified compound is approximately 3-fold lower under the same conditions. 2-hydroxyestradiol was esterified to form 3-hydroxyestra-1,3,5(10)-triene-2,17beta-dioldiacetate. The water solubility of 2-hydroxyestradiol is approximately 0.155 mg/ml at room temperature. The water solubility of the esterified compound is approximately 25-fold lower under the same conditions.

### Example 4

### In Vivo Dissolution of an Estradiol Metabolite Ester

Particles containing 3-benzoyl-2-methoxyestradiol crystals were suspended in 2.5% w/v sodium carboxymethylcellulose and 16 mg was injected into rats subcutaneously. The injection site was dissected after 15 days incubation. Chromatographic analysis of the dissected material showed a release of 6.2%±1.2 of the injected dose.

### Example 5

### Alteration of Estradiol Metabolite Crystal Form

A solution of 2-methoxyestradiol (2ME) (Tetrionics, Madison, Wis.) was made in n-methylpyrrolidone at a 10% w/v concentration. One volume of the 2ME solution was added to 1000 volumes of water stirring rapidly at 23° C. The mixture was allowed to stir prior to the addition of sodium dodecyl sulfate (SDS) to a final concentration of 0.1% w/v. The 2ME was collected by sedimentation and washed twice with 0.1% SDS. Microscopic examination of the sample showed that the recovered crystals were stellate or cruciform with an average diameter of approximately 10 μm. Coulometric Karl Fisher moisture analysis and differential scanning calorimetry showed that the crystal preparation formed was the hemi hydrate of 2ME.

### Example 6

### In Vivo Pharmacokinetics of an Altered Crystal Form of an Estradiol Metabolite

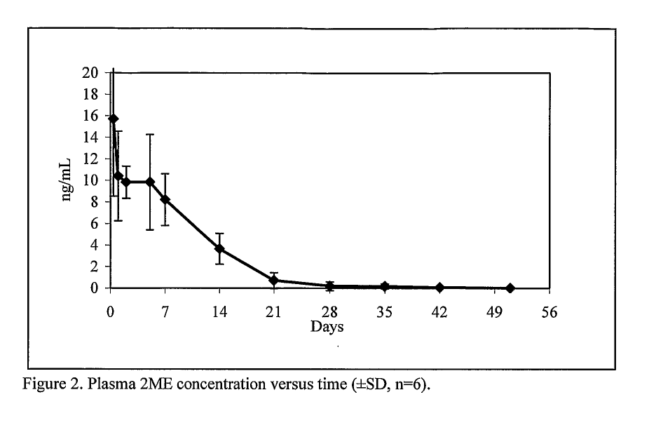

On day 0, each animal in the treatment group (n=6) received 5 mg/rat of 2ME in 0.25 ml injection vehicle consisting of 0.5% w/v sodium carboxymethylcellulose containing 5% w/v mannitol and 0.05% w/v SDS by subcutaneous injection using a 1 cc syringe and a 1 inch, 20-gauge needle. On Day—3 blood was collected from all rats via the tail vein. Rats were bled periodically via the lateral tail vein. Plasma samples were extracted, derivatized, and 2ME concentration was measured using a qualified gas chromatography-mass spectroscopy detection method. Plasma concentrations versus time are plotted in FIG. 2. By day 28, plasma 2ME concentrations had fallen to zero, indicating complete dissolution of the injected dose.

### Example 7

### In Vivo Pharmacokinetics of an Estradiol Metabolite

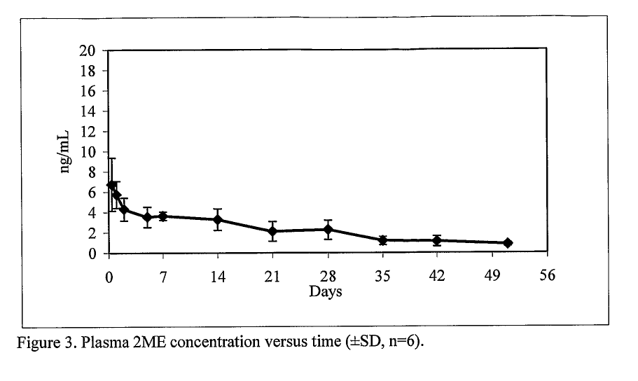

A portion of the 2-methoxyestradiol prepared in Example 1 was ground in a mortar and pestle and sieved through stainless steel screens to generate a population of particles ranging from 90 to 180 micrometers. On day 0, each animal in the treatment group (n=6) received 5 mg/rat of 2ME in 0.25 ml injection vehicle consisting of 0.5% w/v sodium carboxymethylcellulose containing 5% w/v mannitol and 0.05% w/v SDS by subcutaneous injection using a 1 cc syringe and a 1 inch, 20-gauge needle. On Day—3 blood was collected from all rats via the tail vein. Rats were bled periodically via the lateral tail vein. Plasma samples were extracted, derivatized, and 2ME concentration was measured using a qualified gas chromatography-mass spectroscopy detection method. Plasma concentrations versus time are plotted in FIG. 3. The in vivo dissolution was characterized by low initial plasma concentrations for the first week, followed by steady concentrations of approximately 2 to 4 ng/ml between days 7 and 28. 2ME was still measurable in plasma at day 51.

All of the COMPOSITIONS, METHODS and APPARATUS disclosed and claimed herein can be made and executed without undue experimentation in light of the present disclosure. While the compositions and methods of this invention have been described in terms of preferred embodiments, it will be apparent to those of skill in the art that variations may be applied to the COMPOSITIONS, METHODS and APPARATUS and in the steps or in the sequence of steps of the methods described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A composition of comprising;
an estradiol metabolite whereby the in vivo pharmacokinetics of said estradiol metabolite are manipulated by a procedure comprising one or more of chemical modification, crystal packing formation and particle size.

2. The composition of claim 1, wherein said estradiol metabolite further comprises a suspending agent.

3. (canceled)

4. The composition of claim 1, wherein said estradiol metabolite comprises one or more of 2-methoxyestradiol, 2-hydroxyestradiol, 4-methoxyestradiol, and 4-hydroxyestradiol.

5. The composition of claim 1, wherein said estradiol metabolite is a prodrug.

6. The composition of claim 5, wherein said prodrug is an ester.

7. The composition of claim 6, wherein said ester comprises one or more of 3-benzoyl-2-methoxyestradiol; 17-benzoyl-2-methoxyestradiol; 17-acetyl-2-methoxyestradiol; 3-acetyl-2-methoxyestradiol; 3,17-benzoyl-2-methoxyestradiol; 3,17-diacetyl-2-methoxyestradiol; 3-benzoyl-4-methoxyestradiol; 17-benzoyl-4-methoxyestradiol; 17-acetyl-4-methoxyestradiol; 3-acetyl-4-methoxyestradiol; 3,17-dibenzoyl-4-methoxyestradiol; 3,17-diacetyl-4-methoxyestradiol; 3-benzoyl-2-hydroxyestradiol; 17-benzoyl-2-hydroxyestradiol; 17-acetyl-2-hydroxyestradiol; 3-acetyl-2-hydroxyestradiol; 3,17-dibenzoyl-2-hydroxyestradiol; 3,17-diacetyl-2-hydroxyestradiol; 2,3-dibenzoyl-2-hydroxyestradiol; 2,17-dibenzoyl-2-hydroxyestradiol; 2,17-diacetyl-2-hydroxyestradiol; 2,3-diacetyl-2-hydroxyestradiol; 2,3,17-tribenzoyl-2-hydroxyestradiol; 2,3,17-triacetyl-2-hydroxyestradiol; 3-benzoyl-4-hydroxyestradiol; 17-benzoyl-4-hydroxyestradiol; 17-acetyl-4-hydroxyestradiol; 3-acetyl-4-hydroxyestradiol; 3,17-dibenzoyl-4-hydroxyestradiol; 3,17-diacetyl-4-hydroxyestradiol; 3,4-dibenzoyl-4-hydroxyestradiol; 4,17-dibenzoyl-4-hydroxyestradiol; 4,17-diacetyl-4-hydroxyestradiol; 3,4-diacetyl-4-hydroxyestradiol; 3,4,17-tribenzoyl-4-hydroxyestradiol; and 3,4,17-triacetyl-4-hydroxyestradiol.

8. The composition of claim 1, wherein said chemical modification includes derivatization of said estradiol metabolite.

9. The composition of claim 8, wherein said derivative comprises one or more of dicarboxylic acid compounds, diacids, polar compounds, and ionic compounds.

10. The composition of claim 9, wherein such dicarboxylic acid compound comprises one or more of oxalic, malonic, maleic, succinic, glutaric, adipic, pimelic, and pamoic acid.

11. The composition of claim 1, wherein said estradiol metabolite comprises an analog.

12. The composition of claim 11, wherein said estradiol metabolite comprises a prodrug.

13. The composition of claim 1, wherein the estradiol metabolite, estradiol metabolite prodrug, or estradiol metabolite analog has sufficient solubility to achieve the desired steady state tissue concentration within an individual after administration.

14. The composition of claim 1, wherein said estradiol metabolite is in crystalline form.

15. The composition of claim 14, wherein the crystal structure is appropriate to allow sustained in-vivo dissolution of said estradiol metabolite after administration to an individual.

16. The composition of claim 15, wherein the particle size is appropriate to allow sustained in-vivo dissolution of the estradiol metabolite after administration to an individual.

17. The composition of claim 16, wherein the estradiol metabolite has been manipulated by chemical modification, crystal structure, or particle size, or any combination thereof to achieve an appropriate dissolution rate and steady state concentration after administration to an individual.

18. The composition of claim 1, wherein the suspending agent contains a surface active agent.

19. The composition of claim 18, wherein the suspending agent may be chosen from the group consisting of polyvinyl alcohol, polyvinylpyrrolidone tyloxapol, a poloxamer, a polyoxamine dextran lecithin a dialkylester of sodium sulfosuccinic acid, sodium lauryl sulfate, an alkyl aryl polyether sulfonate, a polyoxyethylene sorbitan fatty acid ester, polyethylene glycol, a mixture of sucrose stearate and sucrose distearate, C₁₈H₃₇CH₂(CON(CH₃)CH₂(CHOH)₄(CH₂OH)₂, carboxymethylcellulose calcium, carboxymethylcellulose sodium, methylcellulose, hydroxyethylcellulose, hydroxy propylcellulose, hydroxypropylmethylcellulose phthalate, noncrystalline cellulose, and isononylphenoxypoly(glycidol).

20. The composition of claim 15, wherein the duration of action may be selected to range from a period of three days to a period of one year.

21. The composition of claim 20, wherein the drug, in combination with the suspending agent, is injected into an individual by subcutaneous, subgingival, intramuscular, intraperitoneal, or intraocular routes.

22. The composition of claim 1, wherein said estradiol metabolite comprises 2-methoxyestradiol in a concentration ranging from 20 to 200 mg/ml.

23. (canceled)

24. (canceled)

25. (canceled)

26. (canceled)

27. (canceled)

28. (canceled)

29. (canceled)

30. The composition of claim 1, wherein said manipulation by crystal packing structure comprises one or more of annealing, grinding, or milling to achieve a defined particle size distribution.

31. The composition of claim 30, wherein said composition comprises particles having more than one size distribution.

32. The composition of claim 1, wherein said chemical modification is reversible upon dissolution in a physiological environment.

33. The composition of claim 1, wherein said composition provides a reduction in the symptoms of metabolic disease and endothelin-1 levels.

34. The composition of claim 14, wherein said crystalline form is formed by one or more of thermal treatment, spray drying or antisolvent precipitation.

35. The composition of claim 1, wherein said composition is injectable.

36. The composition of claim 2, wherein the diluent is polyethylene glycol and the estradiol metabolite is soluble.

37. An injectable sustained release composition comprising an estradiol metabolite, wherein the in vivo pharmacokinetics of said composition are manipulated by a procedure comprising one or more of chemical modification, crystal packing formation and particle size.

38. A sustained release composition comprising a crystalline estradiol metabolite.

39. A sustained release formulation containing the composition of claim 1.
